(19) United States
(12) Patent Application Publication
(10) Pub. No.: US 2007/0038180 A1
(43) Pub. Date: Feb. 15, 2007

(54) Apparatus and methods for safe and efficient placement of chest tubes

(76) Inventors: Anil K. Sinha, Lake Jackson, TX (US); Daniel DeOliveira, Lake Jackson, TX (US)

Correspondence Address: OSHA LIANG L.L.P., 1221 MCKINNEY STREET, SUITE 2800, HOUSTON, TX, 77010, US

(21) Appl. No.: 11/503,640

(22) Filed: Aug. 14, 2006

Related U.S. Application Data
(60) Provisional application No. 60/707,661, filed on Aug. 12, 2005.

(51) Int. Cl. A61F 11/00; A61M 5/00

(52) U.S. Cl. 604/117; 606/108

(57) ABSTRACT

An apparatus for placement of a chest tube includes a chest tube assembly and an advancing mechanism configured to move the chest tube assembly. The chest tube assembly includes an outer structure and a chest tube disposed in the outer structure. The chest tube assembly may further include a trocar. The advancing mechanism may include a depth control. A method for using an apparatus for placement of a chest tube includes placing the apparatus against the chest, and advancing the chest tube assembly to a desired depth, wherein the apparatus includes a chest tube assembly and an advancing mechanism configured to move the chest tube assembly, wherein the chest tube assembly includes an outer structure and a chest tube disposed in the outer structure.

### CROSS REFERENCE TO RELATED APPLICATIONS

The application claims the benefits of Provisional Application Ser. No. 60/707,661, filed on Aug. 12, 2005, entitled “Apparatus for Safe and Efficient Placement of a Chest tube for the Treatment of Pneumothorax,” which is incorporated by reference in its entirety. In addition, this application is related to the following two applications filed on the same date: “Self Securing Chest Tubes and Method,” and “Chest Tube Air Counters and Methods.” These two related applications are also incorporated by reference in their entireties.

### BACKGROUND OF INVENTION

1. Field of the Invention

The invention relates generally to apparatus and methods for placement of chest tubes.

2. Background Art

Our lungs are protected by 24 ribs, which also shape and support the chest wall. The parietal pleura is a membrane lining the chest cavity. The visceral pleura is a membrane lining the lungs. The space between these two membranes is known as the pleural space or pleural cavity. This space has a thin film of fluid to provide lubrication and cohesion between the parietal and visceral pleura. A healthy adult has approximately 20 to 25 mL of fluid in the pleural space. Normally, the drainage of this space is regulated by the lymphatic circulation.

Pleural effusion is the accumulation of pathological quantities of fluid in the pleural space. Excess pleural fluid may be caused by liver or kidney failure, congestive heart failure, infection or malignancy blocking the lymphatic system. Four types of fluids may accumulate in the pleural space: blood (hemothorax), serous fluid (hydrothorax), chyle (chylothorax), and pus (pyothorax or empyema). In addition, pneumothorax (collapsed lung) may be caused by trauma or other pathological conditions.

Each of these conditions generally requires the placement of a chest tube to drain the air or fluids placing the patient in danger. The chest tube is a flexible tube that is inserted through the side of the chest into the pleural space. It is used to remove air, fluid, or pus from the pleural space. The free end of the tube is usually attached to an underwater seal, below the level of the chest. This allows air or fluid to escape from the pleural space and prevents anything from returning to the chest. Alternatively, the tube can be attached to a flutter valve to allow patients more mobility.

Chest tubes are usually inserted under local anesthesia. The skin over the area of insertion is first cleansed with antiseptic solution before sterile drapes are placed around the area. The local anesthetic is injected into the skin and down to the muscle. After the area is numb, a passage is made through the skin and muscle into the chest. This is often carried out by performing an incision in the skin with a scalpel blade. Then, the surgeons will use a Kelly clamp to perform blunt dissection of the soft tissues. There is no depth control in the Kelly clamp which may inadvertently go too far inside the pleural cavity. Once the tube is in place it is stitched to the skin to prevent it falling out and a dressing applied to the area. The tube stays in for as long as there is air or fluid to be removed or as long as there is risk of air gathering.

One complication associated with placing the chest tubes arises from lack of depth control during trocar insertion, guidewire insertion, and chest tube insertion. Often, surgeons eyeballs the depth based on experience. This lack of depth control may cause injury, such as puncturing of the lung. Accordingly, there exists a need for new instruments to carry out the placement of chest tubes with depth control.

### SUMMARY OF INVENTION

One aspect of the present invention relates to apparatus for placement of chest tubes. An apparatus in accordance with one embodiment of the invention includes a chest tube assembly and an advancing mechanism configured to move the chest tube assembly. The chest tube assembly includes an outer structure and a chest tube disposed in the outer structure. The advancing mechanism may include a depth control.

Another aspect of the invention relates to methods for using an apparatus for placement of chest tubes. A method in accordance with one embodiment of the invention comprises: placing the apparatus against the chest, and advancing the chest tube assembly to a desired depth, wherein the apparatus comprises: a chest tube assembly having an outer structure and a chest tube disposed in the outer structure, and an advancing mechanism configured to move the chest tube assembly.

Other aspects and advantages of the invention will be apparent from the following description and the appended claims.

### BRIEF DESCRIPTION OF DRAWINGS

FIGS. 1A and 1B show an apparatus in accordance with one embodiment of the invention for placement of a chest tube.

FIGS. 2A-2C show various mechanisms for advancing a chest tube assembly through a chest wall in accordance with embodiments of the present invention.

FIG. 3 shows a chest tube assembly in accordance with one embodiment of the invention.

FIGS. 4A and 4B show two configurations of an outer structure in a chest tube assembly in accordance with embodiments of the invention.

FIG. 5 shows a flow chart of a method for placement of a chest tube in accordance with one embodiment of the invention.

FIG. 6 shows alternative configurations of an apparatus for placement of chest tubes in accordance with embodiments of the invention.

### DETAILED DESCRIPTION

Embodiments of the present invention relate to apparatus and methods for the placement of chest tubes. Embodiments of the invention allow insertion of chest tubes with good control of insertion depth and can prevent inadvertent puncture of lungs.

As noted above, the prior art methods of inserting chest tubes are manually performed, and they rely on surgeons' dexterity and experience. Embodiments of the invention use mechanical and/or electronic devices to facilitate the insertion of chest tubes and to provide insertion depth control.

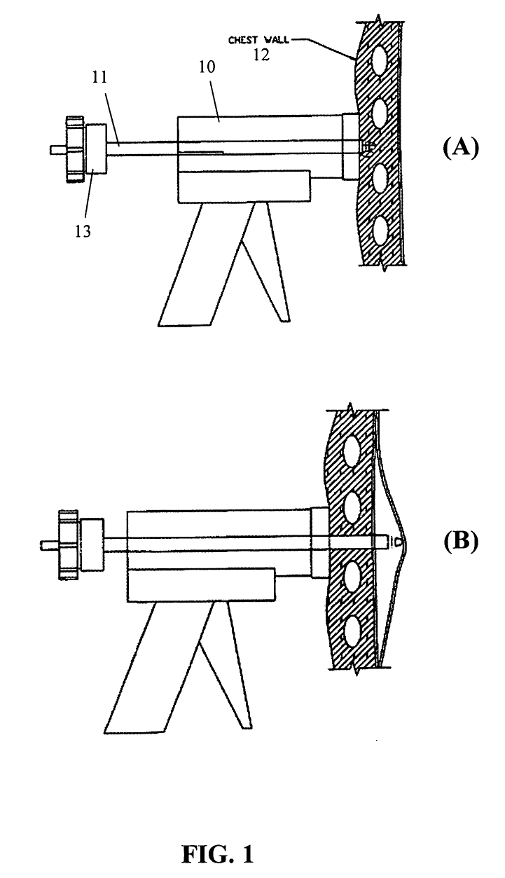

FIGS. 1A and 1B show one embodiment of the invention for inserting a chest tube into a pleural space of a patient. FIG. 1A shows the apparatus 10 with a chest tube assembly 11 pressed against a chest wall 12 of a patient before insertion of the chest tube. FIG. 1B shows the apparatus and the chest tube assembly after insertion of the chest tube assembly into the pleural space.

The embodiment in FIGS. 1A and 1B illustrates a simple mechanical device, similar to a caulking gun, for advancing the chest tube in a controlled manner. With this device, one pulls the trigger to advance the chest tube assembly into the pleural space. Each pull of the trigger may be designed to advance a certain distance, and a maximum number of such advancement may be preset to prevent the chest tube assembly from penetrating too far into the pleural space—to avoid puncturing the lungs. One of ordinary skill in the art would appreciate that various advancement mechanisms may be used with such a device.

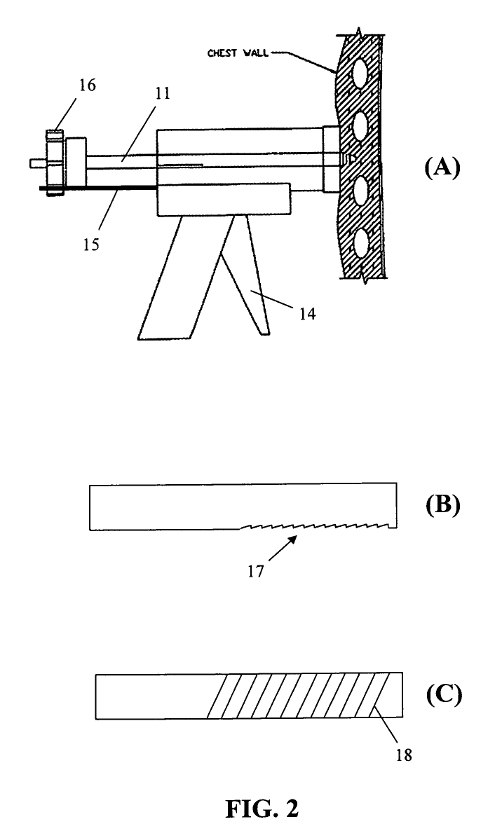

For example, FIG. 2A shows a mechanism similar to a common caulking gun that uses an arm 15 linked to a locking disk 16 for pushing the chest tube assembly 11. FIG. 2B shows an alternative that uses a ratchet mechanism 17, which may be a feature incorporated into the chest tube assembly (e.g., on the outside of the assembly), for the advancement of the chest tube assembly 11. FIG. 2C shows yet another mechanism that uses a screw-like mechanism 18 to rotate and advance the chest tube assembly 11.

When a screw-like mechanism 18 is used, the pulling of the trigger 14 may cause a certain amount of rotation and advancement of the chest tube assembly 11. Alternatively, the apparatus 10 in accordance with embodiments of the invention may not need to have a trigger. Instead, the chest tube assembly 11 may be advanced directly by rotating the chest tube assembly 11, with the aid of a handle (like cork screw for wine bottles) or a nut fixed on the assembly, if necessary. Embodiments of the invention may be mechanically (e.g., using the trigger 14 of FIG. 2A), pneumatically, or electrically driven.

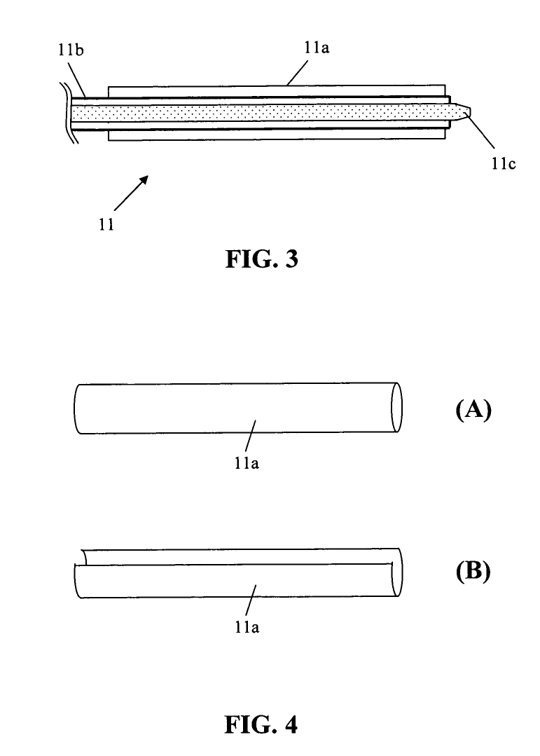

FIG. 3 shows an embodiment of a chest tube assembly of the invention. As shown, the chest tube assembly 11 comprises an outer structure 11a, a chest tube 11b, and a trocar 11c. The outer structure 11a is a hollow structure (e.g., a tube) that is made of a rigid material to provide rigidity necessary for the assembly to penetrate the chest wall. Suitable materials may include, for example, metal, alloy, composite, plastic, polymer, and the like. As noted above with reference to FIG. 2, this outer structure 11a may include a ratchet-like structure (17 in FIG. 2B) or a screw-type structure (18 in FIG. 2C) to facilitate the advancement of the chest tube assembly 11.

The chest tube 11b is typically a flexible tube. It may be made of any material that is used in the art, such as plastic, elastomer, composite, and the like. The trocar 11c, which helps pierce the skin and muscle on the chest wall, may be made of a rigid material, such as metal, alloy, composite, plastic, polymer, and the like.

The chest tube assembly 11 shown in FIG. 3 comprises an outer structure 11a, a chest tube 11b, and a trocar 11c. However, in some embodiments, the trocar 11c may not be necessary, if the tube diameter is small (such as those for use in small children).

Furthermore, while the outer structure 11a is generally tubular shaped, it need not be a tube, as shown in FIG. 4A. In accordance with some embodiments of the invention, the outer structure 11a may have an opening (or slot) along the length of the structure, as shown in FIG. 4B, to facilitate removal of the outer structure 11a from the chest tube 11b after insertion. The opening or slot may comprise a section of the circumference in the cross section. Whether the outer structure 11a is a tube (FIG. 4A) or a slotted tube (FIG. 4B), it may be referred to as having a generally tubular shape.

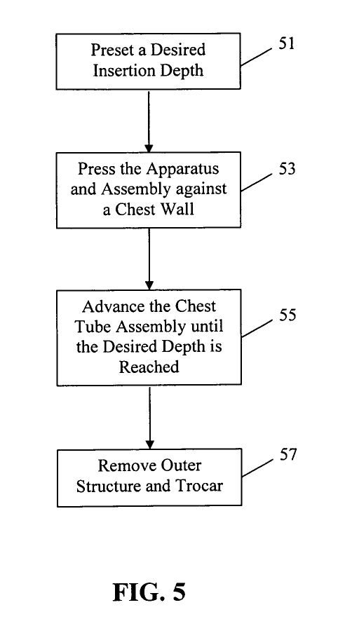

An apparatus of the invention for placing chest tubes may be used to insert a chest tube into a pleural space with reduced risk of injuries to the patient. As shown in FIG. 5, a method in accordance with one embodiment of the invention for placing a chest tube may comprise the steps of: pre-set a desired depth of insertion (step 51) using an apparatus of the invention, the apparatus (such as 10 in FIG. 1) together with the chest tube assembly (such as 11 in FIG. 1); pressing the apparatus and the chest tube assembly against a chest wall (shown as 12 in FIG. 1) (step 53); and advancing the chest tube until the desired depth is reached (step 55). Finally, the outer structure (11a in FIG. 3) and the trocar (11c in FIG. 3) of the chest tube assembly 11 are removed, leaving the chest tube (11b in FIG. 3) in place (step 57).

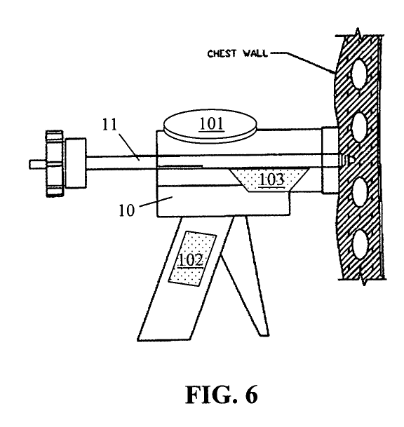

Note that in accordance with other embodiments of the invention, some of these steps may be optional and/or may be performed in a different order. For example, the presetting the desired depth (step 51) may be performed after the pressing against the chest wall (step 53). Furthermore, in some embodiments, it is not necessary to preset the desired depth because this depth may be determined on the flight based on resistance changes. Because embodiments of the invention can advance the chest tube assembly with substantially even forces, it becomes possible to assess the resistance changes, which can then be used to judge whether the chest wall has been penetrated. Therefore, according to some embodiments of the invention, the apparatus 10 may include a mechanism (shown as 103 in FIG. 6) that will automatically lock the chest tube assembly 11 such that it cannot advance further once a sudden reduction in resistance is detected.

In accordance with embodiments of the invention, the trocar 11c need not have a very sharp end because the apparatus of the invention can apply even forces in a consistent manner. A less-sharp tip on the trocar 11c can minimize the possibility of injuries to the patients.

Embodiments of the invention described above are for illustration only. One of ordinary skill in the art would appreciate that various modifications are possible. For example, FIG. 6 shows some of such modifications. As shown in FIG. 6, in accordance with some embodiments of the invention, the apparatus 10 may include a control 101, which may be electronic or mechanical. The control 101 may be used to enter the desired depth of penetration and/or to automatically control the depth of penetration. The control 101 may, for example, include a key pad for entering the desired depth. Alternatively, it may have a dial (e.g., a mechanical device) for entering and controlling the desired depth. If the control 101 is used in an automatic mode, the apparatus 10 may further include an electronic module 102 that may have a motor for advancing the chest tube assembly 11. The electronic module 102 may include its own power source (e.g., a battery) or use an external power source.

Embodiments of the invention provide depth control in the placement of chest tubes by providing substantially even, consistent during the advancement of a chest tube assembly. In addition, one can preset the desired depth, for example by using a locking mechanism (shown as 13 in FIG. 1A), such that the chest tube will not over penetrate into the pleural space. Note that while the locking mechanism 13 in FIG. 1A is shown outside the apparatus body, one of ordinary skill in the art would appreciate that other variations are possible, including having the locking mechanism inside the apparatus.

Furthermore, some embodiments of the invention may include an automatic or backup locking mechanism to further enhance the safety of chest tube insertion. For example, an apparatus in accordance with embodiments of the invention may include a module 103 for detecting the resistance during the advancement of the chest tube assembly 11. Resistance detection can be based on any technique known to one skill in the art. In addition, the module 103 may include a mechanism that will lock the chest tube assembly to prevent it from further advancement once a sudden drop in resistance is detected. This will further enhance the safety of chest tube placement. The locking mechanism may be based on any mechanism known to one skill in the art, such as braking or clamping of the chest tube assembly, or disengagement of the drive mechanism.

Advantages of the present invention may include one or more of the following. Embodiments of the invention provide for apparatus that are capable of safe insertion of chest tubes because they provide good depth control. This will reduce the possibility of injuring the patients. Furthermore, the blunt trocar in accordance with some embodiments of the invention may help prevent unwanted puncture injury, which in conjunction with the strict depth control prevents over-penetration which can damage neighboring organs, especially the lungs.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

What is claimed is:

1. An apparatus for placement of a chest tube, comprising:
a chest tube assembly comprising:
an outer structure; and
a chest tube disposed in the outer structure ; and
an advancing mechanism configured to move the chest tube assembly.

2. The apparatus of claim 1, wherein the chest tube assembly further comprising a trocar disposed in the chest tube.

3. The apparatus of claim 2, wherein the trocar has a blunt tip.

4. The apparatus of claim 1, wherein the advancing mechanism comprises a depth control mechanism.

5. The apparatus of claim 4, wherein the depth control mechanism is activated by a decrease in resistance during the placement to prevent the chest tube assembly from further advancement.

6. The apparatus of claim 1, wherein the advancing mechanism uses a ratchet mechanism or a screw mechanism.

7. The apparatus of claim 1, wherein the outer structure has an opening along a length of the outer structure.

8. The apparatus of claim 1, wherein the outer structure comprises at least one selected from metal, alloy, plastic, polymer, and composite material.

9. The apparatus of claim 1, wherein the depth control mechanism comprises a control device for entering a depth parameter.

10. A method for using an apparatus for placement of a chest tube, comprising:
placing the apparatus against the chest, the apparatus comprising:
a chest tube assembly comprising:
an outer structure, and
a chest tube disposed in the outer structure; and
an advancing mechanism configured to move the chest tube assembly; and
advancing the chest tube assembly to a desired depth.

11. The method of claim 10, wherein the chest tube assembly further comprises a trocar.

12. The method of claim 11, wherein the trocar has a blunt tip.

13. The method of claim 10, wherein the advancing mechanism comprises a depth control mechanism.

14. The method of claim 13, wherein the depth control mechanism is activated by a decrease in resistance during the placement.

15. The method of claim 10, wherein the advancing mechanism uses a ratchet mechanism or a screw mechanism.

16. The method of claim 10, wherein the outer structure has an opening along a length of the outer structure.

17. The method of claim 10, wherein the outer structure comprises at least one selected from metal, alloy, plastic, polymer, and composite material.

18. The method of claim 10, wherein the depth control mechanism comprises a control device for entering a depth parameter.
